(19) United States
(12) Patent Application Publication
(10) Pub. No.: US 2009/0149415 A1
(43) Pub. Date: Jun. 11, 2009

(54) Compounds for modulating the glycolysis enzyme complex and/or the transaminase complex

(75) Inventor: Hans Scheefers, Wettenberg-Wissmar (DE)

Correspondence Address: MAYER & WILLIAMS PC, 251 NORTH AVENUE WEST, 2ND FLOOR, WESTFIELD, NJ, 07090, US

(73) Assignee: Schebo.Biotech AG, 35394 Giessen (DE)

(21) Appl. No.: 10/581,842

(22) PCT Filed: Dec. 6, 2004
(86) PCT No.: PCT/DE2004/002691

§ 371 Date: Oct. 1, 2008

(30) Foreign Application Priority Data
Dec. 5, 2003 (DE) 103 57 301.1

(51) Int. Cl. A61K 31/4035; C07D 209/48; C07D 401/04; A61K 31/7076; A61P 35/00; A61K 31/506; C07H 19/16

(52) U.S. Cl. 514/45; 548/475; 544/333; 536/27.5; 514/417; 514/256

(57) ABSTRACT

The invention relates to compounds for modulating the glycolosis enzyme complex and the transaminase complex, to pharmaceutical compositions containing said compounds, and to the uses of said compounds for the production of pharmaceutical compositions for treating different diseases.

### FIELD OF THE INVENTION

The invention relates to compounds for modulating the glycolosis enzyme complex and/or the transaminase complex and consequently in particular for inhibiting the growth of cells and/or bacteria, to pharmaceutical compositions containing said compounds, and to the uses of said compounds for the production of pharmaceutical compositions for treating different diseases.

### BACKGROUND OF THE INVENTION

Cancer is today one of the most frequent causes of death, and the number of cancer cases in the industrialized countries is continuously growing. This is mainly based on that malignant tumors are diseases of the higher age, and thanks to the successful control of infection diseases, more people now reach this age. In spite of all progress in the diagnostic and therapeutic field, the chances of healing for the most frequent inner cancer forms are rarely higher than 20%. At present, a carcinoma can be destroyed or inhibited in its growth. A back transformation of a tumor cell into a normal cell cannot be achieved yet. The most important therapeutic measures, the operation and the irradiation, remove cancer cells from the organism. Further, the common chemotherapeutic agents used today for cancer, the cytostatics, merely lead to a destruction or damaging of tumor cells. The effect is so little specific, in most cases, that there occur simultaneously severe damages of healthy cells.

In general, tumor cells have a metabolism differing from healthy cells, in particular glycolysis. For instance, a modification of the isoenzyme system involved in the glycolysis and a modification of the transport of NADH are typical for tumor cells. Amongst others, the activity of the enzymes of the glycolysis is increased. This also permits high volumes under the aerobic condition being typical for tumor cells. For details, reference is made to E. Eigenbrodt et al., Biochemical and Molecular Aspects of Selected Cancers, vol. 2, p. 311 ff, 1994.

Various other diseases named below are also accompanied by an (excessive) metabolization by the glycolysis enzyme complex and can be treated by the reduction or inhibition thereof.

### PRIOR ART

>From the document E. Eigenbrodt et al., Biochemical and Molecular Aspects of Selected Cancers, vol. 2, p. 311 ff, 1994, it is known in the art to use glucose analogs for the inhibition of the glycolysis. Other approaches known therefrom are the use of inhibitors of glycolytic isoenzymes, for instance by suitable complex formation or inhibition of complex formations. As a result, tumor cells are so to speak starved out. It is problematic in the above compounds that many of them are genotoxic and/or not sufficiently specific for tumor cells.

### TECHNICAL OBJECT OF THE INVENTION

It is the technical object of the present invention to provide active ingredients, which are capable to modulate or inhibit the glycolosis enzyme complex and the transaminase complex, in particular to inhibit the proliferation of cancer cells and thus the growth of neoplastic tumors as well as excessive defense reactions of the body, such as septic shock, autoimmune diseases, transplantation rejections as well as acute and chronic inflammatory reactions, and that simultaneously with a small up to none at all cytotoxicity relative to cells with an intact glycolysis enzyme complex or other complex structures. In addition, it is intended to inhibit the growth of unicellular organisms.

### BASICS OF THE INVENTION

For achieving this technical object, the invention teaches compounds according to claim 1 and the uses of said compounds.

For AS may in particular be used residues of the proteinogenic amino acids or of the essential amino acids. As far as a compound according to the invention has an optical activity, the various variants such as L and D forms are also comprised. Corresponding considerations apply for the case of (several) chiral centers.

Particularly suited are compounds according to the invention shown in the figures. Sub-stances according to the invention may be pre-sent in the solution independently from the pH in an ionized form (for instance as —COO in a basic environment or as —NH₃⁺ in an acid environment). Salts such as hydrochlorides may also be formed.

The invention is based on the insight that besides the classical metabolic diseases such as diabetes mellitus, adiposity, there are other diseases, such as cancer, autoimmune diseases and rheumatism, which are caused by metabolic disorders. This explains the strong influence of nutrition on these diseases. A directly measurable biochemical parameter for these metabolic disorders is the increase of the pyruvate kinase type M2 (M2-PK), which increases in the blood of patients of all diseases mentioned above and below. Depending on the respective disease, the M2-PK detectable in the blood of the patients comes from different cells: for cancer from tumor cells, for sepsis from immune cells, for rheumatism from immune and/or synovial cells. In healthy cells, the tetrameric form of M2-PK is found in a highly ordered cytosolic complex, the glycolysis enzyme complex. By the over-activation of oncoproteins, there is an emigration of the M2-PK from the complex, and the typical modifications in the tumor metabolism occur. Simultaneously, the phosphoglyceromutase (PGM) leaves the complex and migrates into another enzyme complex, wherein the cytosolic transaminases are associated (see example 2). This complex is designated therefore transaminase complex The substrate of the PGM, glycerate-3-P, is the pre-stage for the synthesis of the amino acids serine and glycine. Both amino acids are essential for the DNA and phospholipid synthesis. By the invasion of the PGM into the transaminase complex, the synthesis of serine from glutamate and thus the glutaminolysis is activated. The same modifications occur in immune cells, when the immune system fails, such as for instance with rheumatism, sepsis or polytrauma. The integration of the metabolism of different cells in multicellular organisms occurs by organ-specific association of the enzymes in the cytosol: in the muscle for instance by association with contraction proteins. For this reason, the different organs are provided with respectively specific isoenzymes. The disbanding of this order necessarily leads to diseases. Unicellular organisms, such as bacteria or yeasts, which react upon a sufficient offering of nutrition by rampant proliferation, do not have a complex organization of the cytosol. Consequently, sub-stances inhibiting the failing metabolism of multicellular organisms, also inhibit the proliferation of such unicellular organisms.

The invention further teaches the use of a compound according to the invention for the production of a pharmaceutical composition for treating one or several diseases from the group consisting of “cancer, chronic inflammations, asthma, arthritis, osteoarthritis, chronic polyarthritis, rheumatic arthritis, inflammatory bowel disease, degenerative joint diseases, diseases of the rheumatic type with cartilage degradation, sepsis, autoimmune diseases, type I diabetes, Hashimoto's thyroiditis, autoimmune thrombocytopenia, multiple sclerosis, myasthenia gravis, chronically inflammatory bowel diseases, Crohn's disease, uveitis, psoriasis, connective tissue diseases, Goodpasture's syndrome, diseases with disturbed adhesion of leukocytes, cachexia, diseases by increased TNFalpha concentration, diabetes, adiposity, bacterial infections, in particular with resistant bacteria”. The term treatment also includes the prophylaxis.

The invention further teaches a pharmaceutical composition, wherein a compound according to the invention is mixed with one or several physiologically well tolerated auxiliary sub-stances and/or carrier substances and is galenically prepared for the local or systemic administration, in particular orally, parenterally, for the infusion or infundation into a target organ, for the injection (e.g. IV, IM, intracapsular or intralumbar), for the application in tooth pockets (space between tooth root and gingiva).

The invention finally teaches the use of a compound according to the invention for the in vitro inhibition of the glycolysis enzyme complex, in particular of pyruvate kinase, asparaginase, serine dehydratases, transaminases, desaminases and/or glutaminases. In particular, the transamination, the oxidative desamination, the hydrolytic desamination, the eliminating desamination and the reductive desamination are blocked.

It is understood that, if applicable, various stereoisomers may exist for the compounds according to the invention, all of which are subject matter of the invention. The term alkyl includes linear and branched alkyl groups as well as cycloalkyl, if applicable also cycloalkyl groups with linear or branched alkyl substituents. The term aryl also comprises aralkyl groups, and alkyl substituents may be alkyl or cycloalkyl.

Surprisingly, it has been found that compounds according to the invention are capable to competitively inhibit the above members of the glycolysis enzyme complex. For instance, the proliferation of cancer cells in therapeutically relevant concentrations can be inhibited. For the dosage range in question, no cytotoxic effect has to be expected. Based on their pharmacological properties, the compounds according to the invention are also excellently suitable for the treatment and the prophylaxis of the further diseases named above. In the context of the indications for the inhibition of inflammations and antirheumatic effects, it is particularly relevant that the substances according to the invention are non-steroidal substances.

The inhibition of the glycolysis enzyme complex and of the transaminase complex comprises in particular the inhibition of the metabolization and the energy gain from serine, glutamine, glutamate, ornithine, proline, alanine and arginine or from other amino acids of this or other families, but also the synthesis of such amino acids used for the energy production; important energy sources for instance in tumor cells, but also in bacteria and yeasts. The cells, bacteria, or yeasts are so to speak starved out. In detail, substances according to the invention block for instance the following reactions: i) threonine to glycine, ii) threonine to α-amino-β-ketobutyrate, iii) α-amino-β-ketobutyrate to glycine, iv) serine pyridoxalphosphate (PLP) Schiff base to aminoacrylate, in particular the folic acid-dependent serine hydroxymethyltransferase, v) aminoacrylate to pyruvate (by displacement of the balance of the natural hydrolysis of the PLP Schiff base toward the Schiff base), vi) transamination by means of PLP to the synthesis of an amino acid from an oxoacid, in particular of the branched-chain transaminase, the α-ketoglutarate, oxalacetate, 3-hydroxypyruvate and glyoxalate transaminase, the glutamate dehydrogenase. In particular, substances according to the invention inhibit the formation of pyruvate from amino acids. Important is the release of NH₂—OH or CH₃—OH (—H at C or N, if applicable, replaced by other residues, for instance alkyl) by glutaminase, arginase, asparaginase or serine hydroxymethyltransferase. This will lead to an increased specificity, since a feature of tumor cells is a high glutaminase and serine hydroxymethyltransferase activity. NH₂—OH (hydroxylamine, HA) for instance can be phosphorylated by the high pyruvate kinase activities in lieu of the —OH of the phosphate (e.g. of the ADP). This will lead to a decoupling of the pyruvate kinase reaction in tumor cells. Therefore, the invention also comprises in all generality all natural metabolites of the substances according to the invention, in particular the aminooxyacetate, i.e. fragments of these substances.

In the transaminase complex are associated, besides the PGM and NDPK, the cytosolic isoforms of the glutamate oxalacetate transaminase (GOT), glutamate pyruvate transaminase (GPT), glutamate dehydrogenase (GIDH) and malate dehydrogenase (MDH). GOT and MDH are components of the malate-aspartate shuttle, by means of which the hydrogen generated in the cytosol is transported into the mitochondria. NAD+ is recycled for the cytosolic glycerinaldehyde-3-phosphate dehydrogenase reaction. The malate-aspartate shuttle is part of the glutaminolysis. For an active malate-aspartate shuttle is important, besides the GOT, the presence of the p36-bound form of the MDH, as shown in Example 3.

For the invention, various further embodiments are possible. For instance, a pharmaceutical composition according to the invention may include several different compounds covered by the above definitions. Further, a pharmaceutical composition according to the invention may in addition contain an effective ingredient differing from the compound of Formula I. Then it is a combination preparation. The various active ingredients used may be prepared in a single preparation form, i.e. the active ingredients are mixed in the preparation form. It is however also possible to prepare the various active ingredients in spatially separated preparation forms of the same or a different type.

As counter ions for ionic compounds according to Formula I can be used Na+, K+, Li+, cyclohexyl ammonium or basic amino acids (e.g. lysine, arginine, ornithine, glutamine).

The drugs produced by means of the compounds according to the invention may be administered orally, intramuscularly, periarticularly, intra-articularly, intravenously, intraperitoneally, subcutaneously or rectally.

The invention further relates to methods for the production of drugs, which are characterized by that at least one compound according to the invention with a pharmaceutically suitable and physiologically well tolerated carrier and, if applicable, further suitable active, additional and auxiliary substances are brought into a suitable preparation form.

Suitable solid or liquid galenic preparation forms are for instance granulates, powders, dragees, tablets, (micro) capsules, suppositories, syrups, juices, suspensions, emulsions, drops or injectable solutions, and preparations with protracted release of active ingredient, for the production of which usual means are used, such as carrier substances, explosives, binding, coating, swelling, sliding or lubricating agents, tasting agents, sweeteners and solution mediators.

As auxiliary substances are named here magnesium carbonate, titanium dioxide, lactose, mannite and other sugars, talcum, milk protein, gelatin, starch, cellulose and their derivatives, animal and vegetable oils such as cod-liver oil, sunflower oil, peanut oil or sesame oil, polyethylene glycols and solvents, such as sterile water and mono or multi-valent alcohols, for instance glycerin.

Preferably, the drugs are produced and administered in dosage units, and every unit contains as the active component a defined dose of the compound according to Formula I of the invention. For solid dosage units such as tablets, capsules, dragees, or suppositories, this dosage may be 1 to 1,000 mg, preferably 50 to 300 mg, and for injection solutions in ampule form, it may be 0.3 to 300 mg, preferably 10 to 100 mg.

For the treatment of an adult with 50 to 100 kg, for instance 70 kg body weight, daily dosages of for instance 20 to 1,000 mg active ingredient, preferably 100 to 500 mg, are indicated. Under certain conditions, higher or lower daily dosages may however also be suitable. The administration of the daily dosage may be made either once in the form of a single dosage unit or in several smaller dosage units as well as repeated administration of subdivided dosages in certain intervals.

Compounds according to the invention are easily synthesizable by the average chemist due to their simple chemical structure.

A pharmaceutical composition according to the invention is for instance prepared for oral administration, for instance with the following auxiliary and carrier substances: colloidal SiO₂, crospovidone, hydroxypropylmethyl cellulose, lactose monohydrate, magnesium stearate, polyethyleneglycol, povidone, starch, talcum, TiO₂, and/or yellow iron oxide. The daily dosage is 1 to 50 mg, preferably 10 to 30 mg. It may be recommendable to administer at the beginning of a therapy an initial dosage of 20 to 500 mg, in particular 50 to 150 mg, for the first 1 to 10 days, in particular the first 1 to 3 days.

In another embodiment, the substance mentioned above is combined with one or several sugar phosphates, for instance fructose-1,6-bisphosphate, glycerate-2,3-bisphosphate, glycerate-3-phosphate, ribose-1,5-bisphosphate, ribulose-1,5-bisphosphate, and the combination of substances may be mixed in one preparation form, for instance a tablet. It is however also possible to provide the components in identical or different preparation forms. The sugar phosphate may be administered in a dosage of 20 to 5,000 mg per day, for instance 100 to 500 mg.

These variants of the invention surprisingly lead to the inhibition of the growth of tumor cells and tumor tissue, since these substances or the metabolite, respectively, bind to the pyruvate kinase and can inhibit or transform back the failing energy metabolism. From this context, there follows as a particular advantage that these substances specifically affect the metabolism of tumor cells and not or to a lower degree the one of normal cells, and that there are thus only minor side effects, if at all.

The efficiency of these substances is surprising, since the known effect as a pyrimidine synthesis inhibitor relates to a completely different functional mechanism, and the phenomenological observation of an antiproliferative effect is substantially directed to immune cells and cells being involved in inflammatory diseases.

Of a particular importance is further a combination of one or several active ingredients mentioned above with one or several of the active ingredients mentioned further above or aminooxyacetate (AOA, NH₂-0-CH₂—COOH, salts or esters thereof, for instance C1-C10 alkyl or hydroxyalkyl ester). E.g. AOA is effective in particular for small tumors (<0.1 to 1 cm³) or prevents the generation thereof, in particular the occurrence of metastases, whereas compounds of the Formulas 10 or 11, if applicable in combination with sugar phosphate are effective against the large tumors. The reason for this is the different metabolism in small and large tumors. The above explanations with regard to combinations apply in an analogous manner.

Substances according to the invention can further be used for the production of a pharmaceutical composition for treating heart insufficiency or the chronic cardiac failure (CCF). Amongst these are the variants or grades of NYHA I to NYHA IV as defined by the New York Heart Association (NYHA) classification. All these diseases are based on an acute or chronic incapability of the heart muscle to provide under load or even in a state of rest the amount of blood or the pumping capacity required for the metabolism of the organism. The reasons for this are the insufficient glycolysis by glucose deficiency in the heart muscle and/or the insufficient oxygen supply thereof and complex coronary inflammation processes (activation of cells of the immune system and complement). This aspect of the invention is based on the insight that by the substances according to the invention, alternative energy-generating biochemical processes are modulated, and that is thus also possible to provide so to speak spare paths for the above imperfectly functioning processes, for instance by activation of the serinolysis or glutaminolysis, or to displace by the substances according to the invention the existing dynamic balance between the glycolysis on the one hand and the glutaminolysis on the other hand in favor of the glycolysis under simultaneous administration of oxygen (increase of the oxygen partial pressure in the blood, for instance by respiration). In this context, the administration of anti-inflammatory substances according to the invention can prevent the life-threatening acidosis (by lactate generation). Compared to prior art measures, such as administration of ACE inhibitors, diuretics, digitalis, positive inotropic substances, or isosorbide dinitrate, the substances according to the invention are immediately involved in the energy metabolism, and the latter is improved. Side effects are consequently comparatively low.

In the context of the present invention, it has been found that at least in the cases of the NYHA grade II to IV, the concentration of tumor M2-PK (=M2-PK dimeric in contrast to normal-M2-PK tetrameric) in cells and/or in the blood increases, which can easily be determined as a routine, different from up to now conventional methods. Therefore, the invention further teaches the use of a tumor M2-PK-detecting test system for the production of a diagnostic agent for the in vitro diagnosis of a heart insufficiency, in particular also of the grade or of the inflammation processes connected therewith. If for a patient, compared to standard values (defined maximum limits; normal collective), increased tumor M2-PK values (collective of the diseased persons) are found in the blood plasma, then this indicates the presence of a heart insufficiency and/or inflammatory processes correlated therewith, at least however the risk to fall ill with heart insufficiency. Such a blood plasma analysis can be performed easily and at short notice. Compared thereto, previous standard methods (gold standard, blood gas analysis) are not suitable for routine tests, and are expensive. For this aspect of the invention, any known test systems can be used, which detect tumor M2-PK, e.g. immunological test systems with antibodies. In particular, per se known test systems, too, may be used, detect tumor M2-PK as a tumor metabolism marker, for instance monoclonal antibodies being specific therefor.

Various substances to be used according to the invention are shown in the further figures. In particular the essential possibilities of variation are shown in an exemplary manner, and the permutations obvious therefrom are not shown for the sake of simplicity. The invention finally comprises all natural metabolites of the described substances. Finally, glycerate-2,3-biphosphate and fructose-1,6-bisphosphate also belong to the substances to be used according to the invention.

In the following, the invention is explained by reference to examples representing potential embodiments only.

### EXAMPLE 1

### Quantification of the Effectiveness of a Compound According to the Invention

Usable Novikoff hepatoma cells can be obtained from the tumor bank of the Deutsches Krebsforschungszentrum, Heidelberg, (Cancer Research 1951, 17, 1010) 100,000 cells each per 25 cm² culture flask are sown out. A substance according to the invention, dissolved in a solvent suitable for use in cell cultures, for instance water, diluted ethanol, dimethylsulfoxide or similar, is added in an increasing concentration to the culture medium, e.g. in the concentration range from 80 μM to 5,000 μM or from 100 μM to 300 μM. After four days of cultivation, the number of cells per flask is counted. In comparison to a control samples (without addition of a compound according to the invention or instead with addition of a reference compound), the measure and the dosage dependence of a proliferation inhibition of the used compound can be seen.

### EXAMPLE 2

### Emigration of the PGM

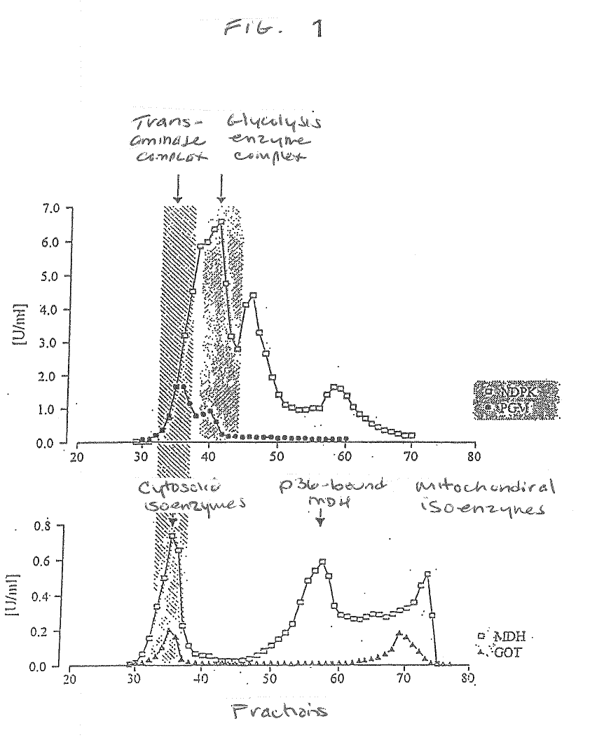

In FIG. 1a, an isoelectric focussing representation of a tumor cell extract (MCF-7 cells) is shown. It can be seen that PGM leaves the glycolysis enzyme complex and migrates into a complex associated with the cytosolic transaminases, the transaminase complex. The transaminase complex is composed as follows: cytosolic glutamate-oxalacetate transaminase (GOT), c-malate dehydrogenase (MDH), phosphoglyceromutase (PGM). Not shown are: c-glutamate-pyruvate transaminase (GPT), c-glutamate-hydroxypyruvate transaminase, c-alanine-hydroxypyruvate transaminase, c-serine-hydroxymethyl transferase and c-glutamate-dehydrogenase (GIDH). The PGM and the nucleotide-diphosphate kinase (NDPK) can be associated in the transaminase complex as well as in the glycolysis enzyme complex.

### EXAMPLE 3

In the FIG. 2 ff., there are shown in an exemplary manner only a series of possibilities of variations of structures according to the invention. Further, various possibilities of permutations can be seen therefrom. The respective possibilities of variations may also be provided for the other possibilities of variations. In principle, the residues of claim 1 can be varied in any way and independently from each other, as indicated there. Simple variants, such as C1 alkyl, C2 alkyl, C3 alkyl, etc. are not shown, and insofar reference is made to the patent claims. Finally, glycerate-2,3-bisphosphate and fructose-1,6-bisphosphate belong to the substances usable according to the invention. Substances according to the invention are further CH₃— (CO)—NHal-CH₂—CH₂—S-Cx alkyl (x=1, 2, 3, 4, 5), wherein S may be replaced by NH.

### EXAMPLE 4

### Synthesis Paths for the Substitution of the Hydroxy Group of a Hydroxy-Amino Acid by the Oxyamino Group

5-hydroxy-2-aminopentane acid is first reacted with t-butyloxycarbonyl azide (t-butyl)-O—(CO)—N₃), which represents a protective group for the amino group. The product is then reacted with benzylbromide, represents a protective group for the amino carboxyl group. The product thus obtained is then reacted with benzhydroxam acid (benz-(CO)—NH—OH), and the desired C—O—N structure is formed under separation of water. In the acid state, this intermediate product is decomposed to 5-oxyamino-2-aminopentane, and the protective groups are also removed.

In a corresponding manner, various derivatives having differently long alkyl chains can be produced. Using for instance 3-hydroxy-2-aminopropane acid results in the compound of the Formula XVI in FIG. 3, 3-oxyamino-2-aminopropane acid, which is the oxyamine derivative of the serine. 3-oxyamino-2-aminopropane acid can alternatively be produced by acid breakdown (2HCl) of cycloserine.

4-oxyamino-2-aminobutane acid can be produced by acid breakdown (HCl) of cyclohomoserine.

### EXAMPLE 5

### Synthesis Paths for the Production of Amino Acid Derivatives, Wherein the Amino Group is Substituted by the Oxyamino Group

As educts can be used arbitrary alpha-hydroxycarbon acids. At the alpha C atom, arbitrary residues can be provided, if applicable with protective groups. Residues may for instance in particular be all amino acid residues.

The educt is first reacted with benzylbromide, which represents a protective group for the carboxyl group. The thus obtained product is then reacted with benzhydroxam acid (benz-(CO)—NH—OH), and the desired C—ON structure is formed under separation of water. In the acid state, this intermediate product is decomposed to alpha-oxyamino carbon acid, and the protective groups are also removed.

Alternatively, the following can be made: The educt is first reacted with benzylbromide, which represents a protective group for the carboxyl group. The thus obtained product is then reacted with 3,3′-di-t-butyloxaziridine, and the desired C—O—N structure is immediately formed. In the acid state, this intermediate product is decomposed to alpha-oxyamino carbon acid, and the protective groups are removed.

### EXAMPLE 6

### Synthesis of Oxyamino Carbon Acids

As the educt, an arbitrary hydroxycarbon acid, for instance with 2 to 7 C atoms, is first reacted with benzylbromide, which represents a protective group for the carboxyl group. The thus obtained product is then reacted with benzhydroxam acid (benz-(CO)—NH—OH), and the desired C—O—N structure is formed under separation of water. In the acid state, this intermediate product is decomposed to alpha-oxyamino carbon acid, and the protective groups are also removed.

### EXAMPLE 7

### Synthesis of an Oxyaminobenzyl Derivative

A para-fluorobenzyl compound is reacted with a hydroxyamino compound. The product therefrom is reacted with N₂H₄ to the desired oxyaminobenzyl derivative (or O-phenylhydroxylamin derivative). The para substituent of the educt is maintained. If applicable, before that protective groups have to be provided for this substituent.

### EXAMPLE 8

### Preferred Variants of the Invention

By the Formula of claim 1 are covered in particular derivatives of natural amino acids, wherein the alpha amino group is replaced by an oxyamino group (—O—NH₂) (X═Y═H; r=1; R1=(CR₂₀R₂₀)ₙ₁—(CO)ᵣ₁—(CR₂₀R₂₀)ₙ₂—(O)ᵣ₂—R₂₀ with n1=1, r1=n2=r2=0; one R₂₀=-Am and the other one R₂₀═—COOH, wherein Am is the residue of an amino acid, which is bound to the alpha-C of an amino acid). These are alpha-oxyamino carbon acids. Basic amino acids may be in particular alanine, serine, cysteine, glutamine acid and asparagine acid. If such a substance has a —OH, —NH₂, —SH, or —COOH functionality in Am, these may in addition be replaced independently from each other by —ONH₂ or —CN functionality. It is preferred for the tumor treatment a combination preparation from the above oxyamino derivatives of the amino acids alanine, serine and/or glutamine acid, an in an arbitrary combination two of these derivatives or all three of them may be provided.

Independently therefrom, substances according to the invention may also be used, besides the indications named in claim 2, for the treatment of tuberculosis and of the sleeping sickness.

1. A compound according to Formula I or a physiologically well tolerated salt thereof 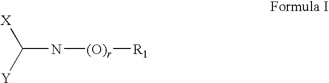
wherein X and Y may be identical or different, and may be -Hal, —H, —O—R₁₀, —NH₂, —(CR₂₀R₂₀)ₙ—O—NH₂, Cₙ alkyl (saturated, single or multiple unsaturated), Cₙ aryl, —NO₂,
wherein R₁₀ may be —H, Cₙ alkyl (saturated, single or multiple unsaturated), Cₙ aryl,
wherein R₁ may be —H, Cₙ alkyl (saturated, single or multiple unsaturated), Cₙ aryl, —CH(ONXY)₂, —C(ONXY)₃, —(CR₂₀R₂₀)ₙ—(CO)ᵣ—(CR₂₀R₂₀)ₙ—(O)ᵣ—R₂₀, —(CR₂₀R₂₀)ₙ—CR₁₁₀—R₁₁₁, —(CR₂₀R₂₀)ₙ—NXY, —SO₂—R₂₀, —O—R₂₀, —(CR₂₀R₂₀)ₙ—(O)ᵣ—(CO)ᵣ—R₁₁₀,
wherein R₁₁₀ may be ═O, -Hal, —COOH, —CN, —SCN, —CNS, —CNO, —N═N—H, —O—CN, —(CO)—CN, —N═N,
wherein R₁₁₁ may be identical to R₁₀, —O—R₁₀,
wherein R₂₀ may be respectively independently —H, —OH, -Hal, Cₙ alkyl (saturated, single or multiple unsaturated), Cₙ aryl, -AS, —NXY, -Z, —C(NH₂)—COOH, —(CO)—CN, —COOH, R₁₁₀, benzyl (unsubstituted or -Hal and/or —OH and/or —ONXY and/or Cₙ alkoxy substituted), —PO₃²⁻, —P₂O₅³⁻,
wherein —O— may be replaced respectively independently by —S— or —Se—,
wherein n may be respectively independently every integer number from 0 to 18,
wherein r is respectively independently 0 or 1,
wherein AS represents respectively independently an amino acid residue, which is obtained by removal of the amino group of an amino acid, or the residue of an amino acid, which is bound to the α-C of an amino acid, or COOH—CH—NH₂,
wherein -Hal is —F, —Cl, —Br, or -J,
wherein -Z is a residue according to one of the following Formulas II to V,
wherein —COOH may be replaced respectively independently by —COOR₁₀, —CHO, —CN, —(CO)—NXY, —C (NXY)₂, —CH—O—NXY, —C(OH)—O—NXY, —(CO)—O—NXY, —(CO)—CN,
wherein free valences are bound by —H,
wherein XYN— or XYN—O— in Formula I may be replaced by NC—, NCS—, NCO—, SNC—, ONC—, HN═N—, N═N—.

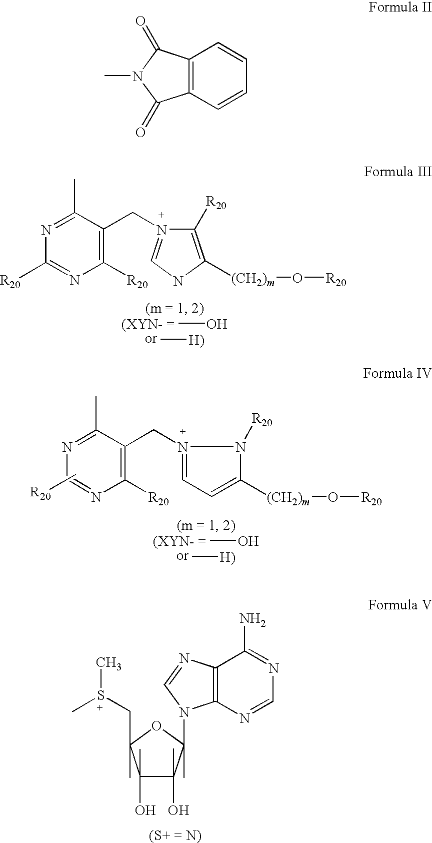

2. The use of a compound according to claim 1 for the production of a pharmaceutical composition for treating one or several different diseases from the group consisting of “cancer, rheumatism, (chronic) inflammations, asthma, arthritis, osteoarthritis, chronic polyarthritis, rheumatic arthritis, inflammatory bowel disease, degenerative joint diseases, diseases of the rheumatic type with cartilage degradation, sepsis, autoimmune diseases, type I diabetes, Hashimoto's thyroiditis, autoimmune thrombocytopenia, multiple sclerosis, myasthenia gravis, chronically inflammatory bowel diseases, Crohn's disease, uveitis, psoriasis, connective tissue diseases, Goodpasture's syndrome, diseases with disturbed adhesion of leukocytes, cachexia, diseases by increased TNFalpha concentration, diabetes, adiposity, bacterial infections, in particular with resistant bacteria (antibiotic), heart insufficiency, chronic cardiac failure (CCF), acidosis”.

3. A pharmaceutical composition, wherein a compound according to claim 1 is mixed with one or several physiologically well tolerated auxiliary and/or carrier substances, and is galenically prepared for the local, in particular oral or systemic, in particular IV administration.

4. The use of a compound according to the claim 1 for the in vitro and/or in viva inhibition of the glycolysis and/or of the glutaminolysis, in particular of pyruvate kinase, asparaginase, serine dehydratases, transaminases, glutamate-oxalacetate transaminase, glutamate-pyruvate transaminase, glutamate-dehydrogenase, malate dehydrogenase, desaminases, and/or glutaminases, in particular in prokaryotes and/or eukaryotes.
